(19) United States
(12) Patent Application Publication
(10) Pub. No.: US 2013/0096523 A1
(43) Pub. Date: Apr. 18, 2013

(54) MULTILAYER BACKING FILM FOR ADHESIVE SKIN BARRIER OF OSTOMY APPLIANCE

(71) Applicants: Moh-Ching Oliver Chang, Lake in the Hills, IL (US); Joel D. Shutt, Gurnee, IL (US)

(72) Inventors: Moh-Ching Oliver Chang, Lake in the Hills, IL (US); Joel D. Shutt, Gurnee, IL (US)

(73) Assignee: HOLLISTER INCORPORATED, Libertyville, IL (US)

(21) Appl. No.: 13/567,455

(22) Filed: Aug. 6, 2012

Related U.S. Application Data
(60) Provisional application No. 61/546,771, filed on Oct. 13, 2011.

(51) Int. Cl. A61F 5/449; A61F 5/448

(52) U.S. Cl. 604/344; 604/338

(57) ABSTRACT

A one-piece ostomy appliance includes an adhesive skin barrier for attaching an ostomy pouch to a user to receive stomal effluent. The adhesive skin barrier is attached to the ostomy pouch via a multilayer backing film, which includes a layer formed of a material having good resistance to chemical ingredients of the adhesive, and a layer formed of a material having good heat sealability to the pouch.

### CROSS-REFERENCE TO RELATED APPLICATION DATA

This application claims the benefit of priority of Provisional U.S. Patent Application Ser. No. 61/546,771, filed Oct. 13, 2011 entitled, MULTILAYER BACKING FILM FOR ADHESIVE SKIN BARRIER OF OSTOMY APPLIANCE.

### BACKGROUND

Ostomy appliances for receiving body waste material, such as ostomy pouches, are known. Such pouches may be used, for example by people who have had surgery such as a colostomy, ileostomy, or urostomy. Such a pouch typically includes opposing sidewalls of flexible polymer film material that are sealed along peripheral edges thereof to define a cavity between the sealed sidewalls. An opening in one of the sidewalls is adapted to receive a stoma of a patient, from which waste material exits the patient and is collected in the pouch cavity.

Ostomy appliances typically include an adhesive barrier for attaching an ostomy pouch to the user. In two-piece ostomy appliances, a connecting flange is provided on the pouch for securing the pouch to an adhesive skin barrier placed to surround the stoma. A mating flange is provided on the skin barrier to secure to the pouch flange. In one-piece ostomy appliances, the adhesive skin barrier is directly attached to the pouch. In such one-piece ostomy appliances, the adhesive skin barrier typically includes a barrier backing film. The barrier backing film is typically heat sealed to a wall of the pouch on one side and laminated to the adhesive on the other side.

Some ostomy pouches, such as Softflex® pouches commercially available from Hollister Inc, include a barrier backing film formed of ethylene-methyl acrylate copolymer (EMA). However, skin barrier adhesives contain petrolatum and tackifiers, which are highly compatible with EMA. Thus, when a barrier backing film formed of EMA is used with such adhesives, the barrier backing film swells and become wrinkled. Further, a barrier backing film formed of polymeric materials having good chemical resistance to petrolatum and tackifiers, such as ethylene-methacrylic acid co-polymer (EMAA) or co-polyester, has insufficient heat sealability to ostomy pouch walls. Thus, there is a need for improved barrier backing films that can provide excellent chemical resistance to petrolatum and tackifiers in the adhesives, and sufficient heat sealability with ostomy pouch walls.

### SUMMARY

Various embodiments of the present disclosure provide a one-piece ostomy appliance adapted to be removably attached to a user for receiving discharge from a stoma of the user. The ostomy appliance includes an adhesive skin barrier for attaching a pouch to the user. The adhesive skin barrier includes a barrier backing film, which provides good heat scalability to the pouch and good resistance against chemical ingredients of the adhesive, such as petrolatum and tackifiers.

In one aspect, an adhesive skin barrier for an ostomy pouch is provided. The adhesive skin barrier includes an adhesive and a multilayer backing film. The backing film includes a first layer formed of a polymeric material having chemical resistance against petrolatum, and a second layer formed of a heat sealable polymeric material. When assembled with an ostomy pouch, the second layer is heat sealed to a wall of the ostomy pouch, while, the first layer is laminated to the adhesive of the skin barrier.

In another aspect, a one-piece ostomy appliance for receiving discharge from a stoma of the patient is provided. The one-piece ostomy appliance includes a pouch and a skin barrier. The pouch includes a first sidewall and a second sidewall, which are sealed together along their peripheral edges to define a collection cavity. Further, an opening is defined by the second sidewall for receiving a. stoma and communicating discharge from the stoma into the collection cavity. The skin barrier is attached to the second sidewall about the opening. The skin barrier includes an adhesive for securing the pouch to a patient and a multilayer backing film. The multilayer backing film includes a first layer, which is directly laminated to the adhesive, and a second layer, which is heat sealed to the second sidewall.

In one embodiment, the multilayer backing film is a two-layer film, of which the first layer is formed of a polymeric material having resistance to petrolatum, and the second layer is formed of a heat sealable polymeric material. For example, the first layer is formed of a polymer selected from the group consisting of a co-polyester, an ethylene-methacrylic acid copolymer (EMAA), and an ionomer, and the second layer is formed of a polymer selected from the group consisting of an ethylene-methyl acrylate copolymer (EMA), an ethylene-vinyl acetate copolymer (EVA), low density polyethylene (LDPE), and linear low density polyethylene (LLDPE).

In another embodiment, the second side wall of the pouch is formed of a polymer selected from the group consisting of EMA, EVA, LDPE, and LLDPE, and the first layer and the second layer of the multilayer backing film are formed of a co-polyester and an EMA, respectively.

Preferably, the multilayer backing film is a two-layer backing film, which is formed by coextruding the first layer and the second layer.

In yet another embodiment, the first layer is formed of an ethylene-methacrylic acid copolymer (EMAA) or an ionomer, and the second layer is formed of EMA or EVA.

In some embodiments, the skin barrier adhesive contains petrolatum and a tackifier, and the first layer of the multilayer backing film does not swell or wrinkle when in contact with the adhesive, such that the skin harrier remains generally wrinkle free during storage and during use.

Other objects, features, and advantages of the disclosure will be apparent from the following description, taken in conjunction with the accompanying sheets of drawings, wherein like numerals refer to like parts, elements, components, steps, and processes.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a cross-sectional view of an ostomy appliance including a pouch and a skin barrier in accordance with an embodiment of the present disclosure; and

FIG. 1A is a partial, enlarged cross-sectional view of the skin barrier and a pouch wall of FIG. 1 showing details of the layers.

### DETAILED DESCRIPTION

While the present device is susceptible of embodiment in various forms, there is shown in the drawings and will hereinafter be described one or more embodiments with the understanding that the present disclosure is to be considered illustrative only and is not intended to limit the device to any specific embodiment described or illustrated. The words “a” or “an” are to be taken to include both the singular and the plural. Conversely, any reference to plural. items shall, where appropriate, include the singular.

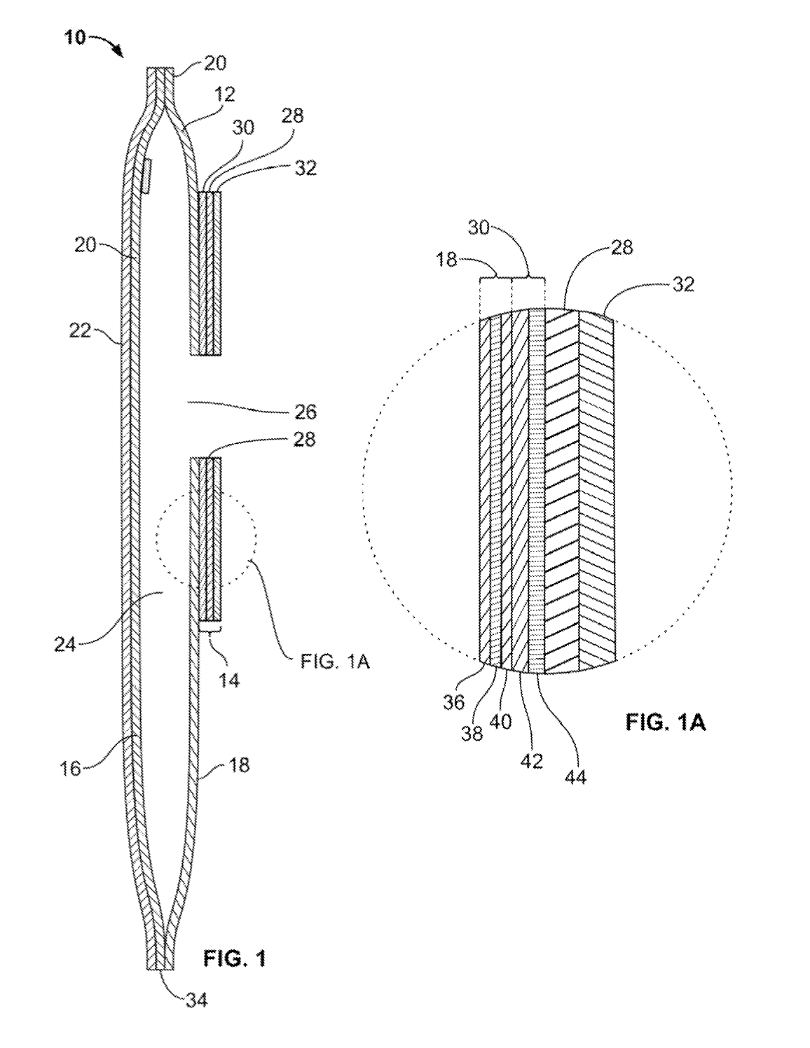

FIG. 1 shows a cross-sectional view of a one-piece ostomy appliance 10 according to an embodiment. The ostomy appliance generally includes a pouch 12 and a skin barrier 14. The pouch 12 includes first and second opposing sidewalls 16, 18, which are sealed around peripheral edges 20 thereof by heat sealing or by any other suitable means to define a cavity 18 therebetween. The pouch 12 also includes a non-woven 22 attached to the first sidewall 16. The non-woven 22 is optional, thus, some embodiments may not include a non-woven. Other embodiments may include the non-woven on one or both walls of the pouch.

The sidewalls 14, 16 can be formed of any suitable material, such as a flexible polymer film that is impermeable to liquids, gasses, and odors. Suitable materials may be single layer films or multilayer films. For example, each of the sidewalls 14, 16 may be formed of a multilayer film having a polyethylene/nylon/polyethylene construction. Each of the sidewalls 12, 14 is about 4-20 mils in thickness.

The pouch 12 includes an aperture or opening 26, which is disposed through the second sidewall 18 and the skin barrier 14 for receiving a stoma (not shown.) The pouch 12 is a single use pouch, which has a closed end 34. Alternatively, the pouch may be a drainable pouch including a closure valve or assembly at a lower portion thereof, which allows for the selective drainage of the contents of the pouch. Various examples of closure means are disclosed, for example, in Jensen U.S. Pat. No. 4,280,498 and Mandzij et al, U.S. Pat. No. 7,879,016, each of which are incorporated herein in its entirety.

The skin barrier 14, which is also referred to herein as a “face plate”, includes an adhesive 28, a backing film 30, and a release liner 32. The adhesive 28 is a medical grade adhesive for attaching to the user's skin around a stoma, such that the pouch 12 is secured about the stoma to accept stoma effluent. The backing film 30 may be formed of a single layer material or a multilayer material. The release line 32 protects the adhesive until it is removed by a user to expose the adhesive for attachment to the peristomal skin.

FIG. 1A shows an enlarged, cross-sectional view of the skin barrier 14, which is attached to the second sidewall 18 of the pouch 12. As shown, the second sidewall 18 is formed of a three-layer film including a first skin layer 36, a barrier layer 38, and a second skin layer 40. The backing film 30 is formed of a two-layer film including a first layer 42 and a second layer 44. The first layer 42 is heat sealed to the second skin layer 40 of the second sidewall 18. Thus, each of the first layer 42 and the second skin layer 40 is formed of a heat sealable material. Suitable heat sealable materials include ethylene-methyl acrylate copolymer (EMA), ethylene-vinyl acetate copolymer (EVA), and other heat sealable polyolefins, such as low density polyethylene (LDPE), linear low density polyethylene (LLPE), etc.

The second layer 44 of the skin barrier 14 is laminated to the adhesive 28. Preferably, the second layer 44 is formed of a material that is resistant to chemical ingredients contained in the adhesive 28. In some embodiments, the adhesive 28 contains petrolatum and tackifiers, which are highly compatible with some heat sealable materials suitable for backing films. When such an adhesive is directly laminated to the backing, film formed of such a heat sealable material, the backing film swells and becomes wrinkled. Thus, the two-layer backing film 30, which includes the heat sealable first layer 42 and the chemical resistant second layer 44 provides for an improved barrier backing film, which can be heat sealed to the pouch with sufficient seal strength and without swelling or wrinkling due to contact with the adhesive 28. Suitable materials for second layer 44 include, but are not limited to, co-polyester, ethylene-methacrylic acid copolymer, and ionomers.

### Example 1

A two-layer backing film includes a first layer and a second layer. As it was with the backing film 30 of FIG. 1A, the first layer is laminated to a skin barrier adhesive, and the second layer is heat sealed to a skin layer of a pouch sidewall, which is formed of EVA. The first layer is formed of co-polyester, such as Hytrel® 4056 by DuPont, and the second layer is formed of EMA, such as EMAC 2205 and EMAC 2207 by Westlake Chemical Corporation of Houston, Tex.

To prepare the two-layer backing film, the co-polyester first layer and the EMA second layer were co-extruded. Surprisingly, it was discovered that the co-extruded co-polyester and EMA layers did not delimitate and provided at least similar seal strength and tensile properties as that of a multilayer film including a tie layer between the EMA layer and the co-polyester layer. Such a two-layer backing film is relatively simple to manufacture and provides cost sayings, as the need for a tie layer is eliminated.

Test results comparing physical properties of a two-layer backing film having a co-polyester layer/EMA layer construction and a three-layer backing film having a co-polyester layer/tie layer/EMA layer construction are provided in Table 1. The three-layer backing film sample included a first layer formed of 97% Hytrel® 4056 and 3% Conpol® 13B (an ethylene-methacrylic acid carrier resin by DuPont) having a thickness of about 1.2 mil; a tie layer formed of Bynel® 21E810 (an anhydride modified ethylene acrylate resin by DuPont) having a thickness of about 0.3 mil; and a third layer formed of EMAC 2205 having a thickness of about 1.0 mil. The two-layer backing film sample included a first layer formed of 97% Hytrel® 4056 and 3% Conpol® 13B having a thickness of about 1.2 mil, and a second layer formed of EMAC 2205 having a thickness of about 1.3 mil. As shown, the two-layer hacking film has physical properties similar to or superior than the three-layer backing film.

TABLE 1

| | | Seal | | Seal | | | | | | | |
|---|---|---|---|---|---|---|---|---|---|---|---|
| | | Strength, | | Strength, | | | | | | | |
| | | MD | | TD | | Tensile | | Elongation- | | Tensile | |
| | Nominal | (lbf/in) | | (lbf/in) | | Mod. | | to-Break | | Strength | |
| | Thickness | | Failure | | Failure | (ksi) | | (%) | | (psi) | |
| Samples | (mil) | Avg | (*a) | Avg | (*a) | MD | TD | MD | TD | MD | TD |
| 3-layer | 2.5 | 4.3 | RBS¹ | 3.0 | RBS | 6.5 | 6.4 | 334 | 560 | 1877 | 1320 |
| 2-layer | 2.5 | 4.5 | RBS | 3.0 | RBS | 7.2 | 7.5 | 322 | 611 | 2063 | 1327 |

¹RBS—ripped (film broken) before seal.

The two-layer backing film has a total thickness of between about 1.5 mil and about 7.5 mil wherein the first layer has a thickness between about 0.5 mil and about 3.5 mil, and the second layer has a thickness between about 0.5 mil and about 5.5 mil. Preferred embodiments of the two-layer backing film are shown in Table 2.

TABLE 2

| | First Layer | Second Layer |
|---|---|---|
| | (adhesive side) | (heat sealing side) |
| Sample# 275-1 | 1.2 mil Hytrel ® 4056 | 1.3 mil EMAC ® 2205 |
| (2.5 mil) | | |
| Sample# 278-1 | 1.2 mil Hytrel ® 4056 | 4.2 mil EMAC ® 2205 |
| (5.4 mil) | | |
| Sample# 278-2 | 1.2 mil Hytrel ® 4056 | 4.2 mil EMAC ® 2207 |
| (5.4 mil) | | |
| Sample# 278-3 | 2.0 mil Hytrel ® 4056 | 3.4 mil EMAC ® 2207 |
| (5.4 mil) | | |

### Example 2

A two-layer backing film includes a first layer and a second layer. As it was with the previously discussed two-layer backing films, the first layer is laminated to a skin barrier adhesive, and the second layer is heat sealed to a skin layer of a pouch sidewall. In this example, the first layer is formed of ethylene-methacrylic acid copolymer (EMAA), such as Nucrel® 0903HC by DuPont, or ionomer such as Surlyn® 1857 (EMAA-Zn) by DuPont; and the second layer is formed of EMA, such as EMAC 2205 by Westlake Chemical Corporation, or EVA, such as Bynel® 3861 (anhydride-modified EVA) by DuPont. Preferred embodiments of such two-layer backing film are shown in Table 3. The sample two-layer films were prepared by co-extruding the first and second layers.

TABLE 3

| | First Layer | Second Layer |
|---|---|---|
| | (adhesive side) | (heat sealing side) |
| Sample#1 | 1.25 mil Nucrel ® 0903HC | 1.25 mil EMAC ® 2205 |
| (2.5 mil) | | |
| Sample#2 | 1.25 mil Surlyn ® 1857 | 1.25 mil EMAC ® 2205 |
| (2.5 mil) | | |
| Sample#3 | 1.5 mil Nucrel ® 0903HC | 0.75 mil Bynel ® 3861 |
| (2.25 mil) | | |
| Sample#4 | 1.25 mil Surlyn ® 1857 | 0.75 mil Bynel ® 3861 |
| (2.25 mil) | | |

### Example 3

A three-layer backing film includes a first layer, a second tie layer and a third. layer. The first layer is laminated to a face plate adhesive, and thus formed of a material having good resistance to chemical ingredients of the adhesive. The third layer is heat sealed to a skin layer of a pouch sidewall, and thus formed of a heat sealable material. The second tie layer is provided to improve compatibility between the first layer and the third layer to enhance adhesion between the layers. Similar to Example 2, the first layer of this example is formed of EMAA, such as Nucrel® 0903HC by DuPont or ionomer, such as Surlyn® 1857 (EMAA-Zn) by DuPont. The second tie layer is formed of anhydride-modified EVA, such as Bynel® 3861 by DuPont. The third layer is formed of EMA, such as EMAC 2205 by Westlake Chemical Corporation. Preferred embodiments of such two-layer backing film are shown in Table 4.

TABLE 4

| | First Layer | Second Layer | Third Layer |
|---|---|---|---|
| | (adhesive side) | (tie layer) | (heat sealing side) |
| Sample#1 | 1 mil Nucrel ® | 0.5 mil Bynel ® | 1 mil EMAC ® |
| (2.5 mil) | 0903HC | 3861 | 2205 |
| Sample#2 | 1 mil Surlyn ® | 0.5 mil Bynel ® | 1 mil EMAC ® |
| (2.5 mil) | 1857 | 3861 | 2205 |

Numerous modifications to the present disclosure will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

We claim:

1. An adhesive skin barrier for an ostomy pouch, comprising:
an adhesive; and
a multilayer backing film, the backing film comprising:
a first layer formed of a polymeric material having chemical resistance against petrolatum; and
a second layer formed of a heat sealable polymeric material,
wherein the adhesive is laminated to the first layer and the second layer is heat sealed to a wall of an ostomy pouch.

2. The skin barrier of claim 1, wherein the first layer is formed of a polymer selected from the group consisting of a co-polyester, an ethylene-methacrylic acid copolymer (EMAA), and an ionomer.

3. The skin barrier of claim 2, wherein the second layer is formed a polymer selected from the group consisting of an ethylene-methyl acrylate copolymer (EMA), an ethylene-vinyl acetate copolymer (EVA), low density polyethylene (LDPE), and linear low density polyethylene (LLDPE).

4. The skin barrier of claim 1, wherein the first layer is formed of a co-polyester and the second layer is formed of an EMA.

5. The skin barrier of claim 1, wherein the first layer is formed of an ethylene-methacrylic acid copolymer (EMAA), and the second layer is formed of EMA or EVA.

6. The skin barrier of claim 1, wherein the first layer is formed of an ionomer, and the second layer is formed of EMA or EVA.

7. The skin barrier of claim 1, wherein the skin barrier is a two-layer film, wherein the first layer and the second layer are coextruded.

8. The skin barrier of claim 1, wherein the skin barrier is a three-layer film, wherein the first layer is formed of an ionomer or an EMAA or a co-polyester, and the second layer is formed of an EMA; the skin barrier further including a tie layer between the first layer and the second layer.

9. The skin barrier of claim 1, wherein the adhesive contains petrolatum and a tackifier, and wherein the first layer has chemical resistance against the petrolatum, the tackifier, and other ingredients in the adhesive, and thus does not swell or wrinkle when in contact with the adhesive, and wherein the skin barrier remains generally wrinkle free during storage and during use.

10. A one-piece ostomy appliance for receiving discharge from a stoma of the patient, comprising:
a pouch including a first sidewall and a second sidewall, the first sidewall and the second sidewall being sealed together along their peripheral edges to define a collection cavity;
an opening defined by the second sidewall for receiving a stoma and communicating discharge from the stoma into the collection cavity; and
a skin barrier attached to the second sidewall about the opening, the skin barrier comprising:
an adhesive for securing the pouch to a patient; and
a multilayer backing film, the multilayer backing film including a first layer and a second layer, wherein the first layer is directly laminated to the adhesive and the second layer is heat sealed to the second sidewall.

11. The ostomy appliance of claim 10, wherein the multilayer backing film is a two-layer film, wherein the first layer is formed of a polymeric material having resistance to petrolatum, and the second layer is formed of a heat sealable polymeric material.

12. The ostomy appliance of any one of claims 10, wherein the first layer is formed of a polymer selected from the group consisting of a co-polyester, an ethylene-methacrylic acid copolymer (EMAA), and an ionomer.

13. The ostomy appliance of claim 10, wherein the second layer is formed a polymer selected from the group consisting of EMA, EVA, LDPE, and LLDPE.

14. The ostomy appliance of claim 10, wherein the second side wall is formed of a polymer selected from the group consisting of EMA, EVA, LDPE and LLDPE, the first layer is formed of a co-polyester and the second layer is formed of an EMA.

15. The ostomy appliance of claim 14, wherein the first layer and the second layer are coextruded to form the multilayer backing film; the multilayer backing film being a two-layer film.

16. The ostomy appliance of claim 10, wherein the first layer is formed of an ethylene-methacrylic acid copolymer (EMAA), and the second layer is formed of EMA or EVA.

17. The ostomy appliance of claim 10, wherein the first layer is formed of an ionomer, and the second layer is formed of EMA or EVA.

18. The ostomy appliance of claim 10, wherein the multilayer backing film is a three-layer film and includes a tie layer arranged between the first layer and the second layer, wherein the first layer is formed of a polymeric material having resistance to petrolatum, and the second layer is formed of a heat sealable polymeric material.

19. The ostomy appliance of claim 10, wherein the adhesive contains petrolatum and a tackifier, and wherein the first layer has chemical resistance against the petrolatum, the tackifier, and other ingredients in the adhesive, and thus does not swell or wrinkle when in contact with the adhesive, and wherein the skin barrier remains generally wrinkle free during storage and during use.
